(19) United States
(12) Patent Application Publication
(10) Pub. No.: US 2022/0010258 A1
(43) Pub. Date: Jan. 13, 2022

(54) DEVICE THAT CAN SERVE AS A HEMATO-ENCEPHALITIC BARRIER MODEL

(71) Applicants: UNIVERSITE DE POITIERS, POITIERS CEDEX (FR); Centre Hospitalier Universitaire de Poitiers, POITIERS (FR)

(72) Inventors: GUYLÈNE PAGE, POITIERS (FR); Hanitriniaina RABEONY, POITIERS (FR); Damien CHASSAING, ST MAURICE LA CLOUERE (FR); Emilie DUGAST, CHAMPAGNE-LE-SEC (FR); Thierry JANET, LIGUGE (FR)

(21) Appl. No.: 16/637,998

(22) PCT Filed: Aug. 10, 2018
(86) PCT No.: PCT/EP2018/071759

§ 371 Date: May 5, 2021

(30) Foreign Application Priority Data
Aug. 10, 2017 (FR) 1757643

(51) Int. Cl. C12M 1/42; C12M 1/00; C12M 1/12; C12N 5/079; C12N 5/071

(52) U.S. Cl. CPC C12M 35/08; C12M 23/34; C12M 25/02; C12N 2502/28; C12N 5/069; C12N 5/0697; C12N 2502/08; C12N 5/0622

(57) ABSTRACT

The present invention relates to a device which can serve as a model of a hemato-encephalic barrier (HEB) comprising two compartments in which certain cell types are arranged. The invention also relates to the method for preparing said device and the use thereof as a model of the HEB.

The present invention relates to a device that can serve as a model of a hemato-encephalic barrier (HEB) comprising two compartments in which certain cell types are arranged.

The brain is separated and isolated from the circulating bloodstream by a particular structure, the hemato-encephalic barrier (HEB or blood-brain barrier (BBB)). This barrier is mainly formed by endothelial cells that interact with the neighbouring cells, in particular pericytes and astrocytes. The latter interact with microglia and neurons. The HEB maintains an environment that serves to enable the proper functioning of neurons by performing several primary functions: finely controlling the passage of molecules and ions, delivering nutrients and oxygen instantaneously as needed by the neurons, and protecting the brain from toxins and pathogens.

In order for medicinal products designed to act on the brain to be effective, they must be able to pass through the HEB easily. In order to study the passage of molecules through the HEB and accordingly enable optimization thereof, several in vitro models of this barrier have been developed to date. These models most often entail the culturing of a number of different cell types in which endothelial cells are separated from other cell types by a porous synthetic membrane.

However, there continues to exist a need for barrier models that are more akin to the HEB in vivo and that can be used to carry out various different studies such as study of the pathophysiology of certain degenerative diseases and disorders, studying the effect of aging on the HEB, and studying the passage of molecules, in particular for therapeutic or diagnostic purposes, etc.

The inventors sought and succeeded in bringing about direct contact between the pericytes and endothelial cells, which thereby promoted the appearance of structures organised into vessels, and thus made it possible to obtain an impermeable model that is very similar to the HEB in vivo.

An object of the invention therefore relates to a device comprising two compartments that are separated by a porous synthetic membrane: one compartment referred to as luminal compartment comprising endothelial cells and pericytes, and one compartment referred to as abluminal compartment comprising astrocytes and microglia. In addition, on the luminal side, peripheral blood mononuclear cells (PBMCs) can be deposited.

This type of device makes it possible to obtain HEB models that exhibit an impermeability and functionality very similar to that observed in vivo. The inventors have even observed that the endothelial cells organise themselves into vessels in this device.

The porous synthetic membrane may be tubular or planar.

The term “luminal compartment” is understood to refer to the compartment formed by the lumen of a tubular device or the upper compartment in a planar device.

The term “abluminal compartment” is understood to refer to the compartment on the exterior of the luminal compartment in a tubular device or the lower compartment in a planar device.

This device comprising the major cellular actors of the HEB replicates the neurovascular microenvironment that forms the HEB in vivo and thus provides the means for taking into account and replicating the multiple cellular and molecular interactions that can occur in vivo.

According to one embodiment, the pericytes and endothelial cells are arranged in superimposed layers in the luminal compartment. In a preferred manner, the pericytes are thus then arranged on or in contact with the porous synthetic membrane and the endothelial cells are arranged above the pericytes, such that the pericytes are in very close contact with the endothelial cells. The seeding ratio of pericytes to endothelial cells may vary, and in particular may be chosen in a manner so as to correspond to the ratio present in the HEB under study, for example the human HEB. Preferably, the seeding ratio of pericytes to endothelial cells is comprised approximately between ½ to ¼ and on a preferred basis is approximately ⅓ (corresponding to the ratio of pericytes to endothelial cells in the human HEB).

According to one specific embodiment, the luminal compartment additionally also comprises blood cells, and in a preferred manner peripheral blood mononuclear cells (PBMCs). These are then arranged above the endothelial cells.

The term “porous synthetic membrane” is understood to refer to a permeable support, which allows for small molecules or ions to pass through, and indeed in one particular embodiment, which allows for cell extensions or cells to pass through, depending on the pore size chosen. This support thus allows the cells of each compartment to interact at a distance. Around the membrane, a cell structure similar to an HEB is progressively put in place, under the combined action of the development of the seeded cells, and this structure, once mature, in addition comprises two extracellular matrices: the vascular basement membrane and the parenchymal basement membrane.

This porous synthetic membrane may be made of polyester (clear support allowing for good visibility of the cells under the microscope) or polycarbonate (translucent support allowing for low visibility of the cells under the microscope). This membrane may be precoated (“coating”) in advance with the constituents of the extracellular matrix, and in particular with collagen, laminin, fibronectin or a mixture of the same depending on the desired applications. The size of pores is to be chosen in accordance with the desired applications. Where it is sought to carry out studies pertaining to permeability, transport of molecules, cell polarity, protein and receptor endocytosis and/or cell interactions, it is preferable to use supports wherein the pore diameter is comprised between about 0.4 μm and about 3 μm and preferably is about 0.4 μm in diameter. Thus, in one embodiment, the support has a pore diameter comprised between about 0.4 and about 3 μm, preferably of about 0.4 μm. For studies seeking to investigate the passage of cells through the support, such as PBMCs for example, it would be preferable to take supports having a pore diameter comprised between about 3 μm and about 8 μm, preferably between about 5 μm and about 8 μm. Thus, in one embodiment, the support has a pore diameter comprised between about 3 μm and about 8 μm, preferably between about 5 μm and about 8 μm.

According to one specific embodiment, the abluminal compartment comprising astrocytes additionally comprises microglia. The seeding ratio of microglia to astrocytes may vary, and in particular may be chosen in a manner so as to correspond to the ratio present in the HEB under study, for example the human HEB. In the embodiment in which astrocytes and microglia are used, in a preferred manner the seeding ratio of microglia to astrocytes is comprised between about 1% and about 10% and is preferably about 5%. Preferably the abluminal compartment comprising astrocytes is free of pericytes.

In a preferred manner all the cells of the device according to the invention originate from the same animal species, in particular from mammals. According to one embodiment of the invention, the cells are rodent cells, on a preferred basis they are mouse cells. According to another embodiment of the invention, the cells are primate cells, on a preferred basis they are human cells.

According to one embodiment, one or more cell types of the device according to the invention are derived from immortal cell cultures, in one particular embodiment all of the cell types are derived from immortal cells.

The term “immortal cells” is understood to refer to immortal cells that are derived from tumours, spontaneously immortal cells and/or cells rendered immortal (“immortalised”) by the introduction of at least one cellular or viral oncogene. According to one specific embodiment, one or more cell types of the device according to the invention are derived from primary cultures of cells rendered immortal by the introduction of at least one viral or cellular oncogene.

In one preferential embodiment, one or more cell types of the device according to the invention are derived from primary cultures, in a preferred manner all of the cell types are derived from primary cultures.

The term “primary culture” is understood to refer to a culture of cells derived directly from the tissue and/or cells of an individual. In a variant embodiment, one or more cell types of the device according to the invention are derived from primary cultures of tissues and/or cells taken from individuals of the same species and of the same age, in a preferred manner all of the cell types are derived from primary cultures of tissues and/or cells taken from individuals of the same species and of the same age. According to one embodiment of the invention, one or more, and preferably all of the cell types are derived from adult individuals. In this manner the device according to the invention also makes it possible to study the variations due to aging or the impact on the HEB of diseases that develop over the life of the individual. Unlike immortalised cells, the cells derived from primary cultures retain contact inhibition, and thus the use of these cells provides the means to limit cell proliferation in the device. In addition, the use of primary cultures further serves to enable the device to more closely approximate in vivo conditions.

The term “individual” is understood to refer to a subject from an animal species, in particular mammals. According to one embodiment of the invention, the one or more individual(s) are rodents, and on a preferred basis mice. In another embodiment of the invention, the individual(s) are primates, and on a preferred basis humans.

In one variant embodiment of the device according to the invention the endothelial cells and the pericytes are derived from primary cultures of mouse cells and in a preferred manner from adult mice. In a particularly preferred manner, the endothelial cells and the pericytes are derived from primary cultures of mouse cells from mice that are at least 3 months old, more particularly at least 6 months old, and in one preferred embodiment at least 12 months old.

Thus according to one variant of the method according to the invention all of the cell types are derived from primary cultures of mouse cells and in a preferred manner from adult mice. In a particularly preferred manner, all of the cell types are derived from primary cultures of mouse cells from mice that are at least 3 months old, more particularly at least 6 months old, and in one preferred embodiment at least 12 months old.

When one or more cell types of the device according to the invention are derived from primary cell cultures, then, according to one particular embodiment, one or more of these cell types are disease model cell types.

The term “disease model cell types” is understood to refer to cell types derived from animal models which replicate the pathologies that appear spontaneously or are induced by means of genetic engineering methods (such as transgenesis) or with pharmacological tools in order to replicate the characteristics of cells of individuals affected by these particular pathologies. By way of examples mention may be made of the cell types derived from mouse models APPswePS1dE9 for Alzheimer's disease, hemi-parkinsonian or parkinsonian models (toxic injections of MPTP, 6-OHDA, or transgenic mice mutated for alpha-synuclein for Parkinson's disease, models of transgenic mice mutated for the huntingtin gene for Huntington's disease, mutation of the gene C9ORF72 for amyoptrophic lateral sclerosis (ALS). Preferably the pathologies according to the invention are pathologies that have or are suspected of having an effect on the HEB such as: neurodegenerative diseases (Alzheimer's, Parkinson's, Huntington's, ALS, etc), cerebrovascular accident, and cerebral cancers.

According to one possible embodiment, the porous synthetic membrane is in the form of a tube, into which a fluid may be introduced, replenished, put into circulation.

According to one preferred embodiment, the porous synthetic membrane is a planar membrane that is horizontally arranged. In this embodiment, the chambers are arranged one above the other.

According to one embodiment, the device can be cryopreserved in order to facilitate its transport or to delay its use.

The object of the invention also relates to a preparation method for preparing the device according to the invention.

According to one embodiment of the invention, this method comprises the following steps:
a) seeding of astrocytes on one surface and/or on one side of the porous synthetic membrane (the side that will become the abluminal side);
b) inserting or depositing of the porous synthetic membrane on the said surface, in a manner such that if the porous synthetic membrane comprises astrocytes, then the side comprising the astrocytes is positioned to be facing the surface;
c) seeding of the luminal side of the porous synthetic membrane with pericytes and endothelial cells.

The step b) thus serves to create the abluminal compartment between the surface and the synthetic porous membrane.

In this embodiment of the invention, the surface is a support on which the device rests. When the porous synthetic membrane is planar, the surface may be, for example, the bottom of a culture dish.

According to another embodiment of the invention, this method comprises the following steps:

a) seeding of astrocytes on one side of the porous synthetic membrane;

b) seeding of the other side of the porous synthetic membrane with endothelial cells and pericytes and optionally;

c) depositing of the porous synthetic membrane on one surface in a manner such that the side comprising astrocytes is positioned to be facing the surface.

According to one embodiment, the method for preparing the device according to the invention comprises an additional step of cryopreservation of the device.

According to one embodiment, the pericytes and endothelial cells are arranged in superimposed layers in the luminal compartment. Preferably in the methods according to the invention the pericytes are seeded before the endothelial cells. In a preferred manner the pericytes are arranged on or in contact with the porous synthetic membrane and the endothelial cells are arranged above the pericytes, the pericytes being consequently in close contact with the endothelial cells. The seeding ratio of pericytes to endothelial cells may vary, and in particular may be chosen in a manner so as to correspond to the ratio present in the HEB under study, for example the human HEB. Preferably, the seeding ratio of pericytes to endothelial cells is comprised about between ½ to ¼ and on a preferred basis is about ⅓ (corresponding to the ratio in the human HEB).

Preferably the porous synthetic membrane is in contact with the cells of each compartment.

According to one specific embodiment, the seeding step for seeding the porous synthetic membrane with pericytes and endothelial cells further comprises the addition of blood cells, and on a preferred basis peripheral blood mononuclear cells (PBMCs). In a preferred manner the addition of blood cells takes place after seeding of the pericytes and endothelial cells.

According to one specific embodiment, the one or more seeding step(s) for seeding with astrocytes also comprise(s) seeding with microglia. Preferably the astrocytes and microglia are cultured together before being seeded in the device. The seeding ratio of microglia to astrocytes may vary, and in particular may be chosen in a manner so as to correspond to the ratio present in the HEB under study, for example the human HEB. Preferably, the seeding ratio of microglia to astrocytes is about 5% (corresponding to the ratio in the human HEB).

Preferably, the one or more seeding step(s) for seeding with astrocytes does not include seeding of pericytes.

On a preferred basis, all of the cells seeded according to the method of the invention originate from the same animal species, in particular from mammals. According to one embodiment of the invention, the cells are rodent cells, on a preferred basis mouse cells. According to another embodiment of the invention, the cells are primate cells, on a preferred basis human cells.

According to one embodiment, one or more cell types of the method according to the invention are derived from cultures of immortal cells. According to one particular embodiment, all of the cell types are derived from immortal cells.

According to one preferential embodiment, one or more cell types of the method according to the invention are derived from primary cultures, on a preferred basis all of the cell types are derived from primary cultures. In one variant, one or more cell types of the method according to the invention are derived from primary cultures of tissues taken from individuals of the same age, on a preferred basis all of the cell types are derived from primary cultures of tissues taken from individuals of the same age. According to one embodiment of the invention, one or more, and preferably all of the cell types are derived from adult individuals.

Thus according to one variant of the method according to the invention the endothelial cells and the pericytes are derived from primary cultures of mouse cells and on a preferred basis from adult mice. In a particularly preferred manner, the endothelial cells and the pericytes are derived from primary cultures of mouse cells from mice that are at least 3 months old, more particularly at least 6 months old, and in one preferred embodiment at least 12 months old.

Thus according to one variant of the method according to the invention all of the cell types are derived from primary cultures of mouse cells and on a preferred basis from adult mice. In a particularly preferred manner, all of the cell types are derived from primary cultures of mouse cells from mice that are at least 3 months old, more particularly at least 6 months old, and in one preferred embodiment at least 12 months old.

When one or more cell types of the method according to the invention are derived from primary cultures, then, according to one particular embodiment, one or more of these cell types are disease model cell types for various pathologies.

The object of the invention also relates to the use of the device according to the invention and in particular the use thereof as a model of the HEB. The object of the invention also relates to the use of the device according to the invention as a pathological HEB model.

In particular the device may be used for testing the permeability of the model. Indeed, the device may be used to study its permeability to:
molecules, in particular therapeutic molecules (in particular small biological or synthetic molecules, antibodies, psychotropic drugs, etc.);
viruses (in particular HIV);
spores (such as bacterial spores, fungal spores, plant spores, etc., for example for the study of fungal infections such as certain meningitis); or even
exosomes (in particular for the study of the propagation of cancers);
liposomes or nanoparticles;
vectorised or naked nucleic acids;
cells (in particular PBMCs, cancer cells, bacteria, etc);

hereinafter referred to as “compounds”.

Thus the invention relates to the use of the device according to the invention in order to test the permeability of the model to a compound, the method comprising the following steps:

a) adding of the said compound to one of the compartments of the device;

b) incubation of the device;

c) detection and analysis of the presence of the said compound and/or its metabolites in the compartment where the addition of the said compound has not taken place; and possibly

d) deducing therefrom the permeability of the model with respect to this compound.

Advantageously, during the step a), a known amount of the compound is added and the step c) serves to enable measurement of the amount of the compound or its metabolites in the compartment where the addition has not taken place. In addition, it is also possible to detect and analyse the presence of the said compound or its metabolites in the compartment where the addition has taken place, in particular in order to determine the quantity of this compound remaining in the said compartment.

Techniques for the detection and analysis of the presence of the compound (or its metabolites) mentioned here above are well known in the state of the art. For example, detection or analysis of the presence of the compound can be performed by various analytical chemistry techniques depending on the compound under study, including HPLC coupled with one or even two mass spectrometry techniques.

Advantageously the compound may be labelled in order to facilitate its detection. Indeed if fluorescent or radiolabelled compounds are available (in particular tracers which would be used in diagnostics and radiolabelled, for example with Fluorine 18 or Carbon 11), fluorescence intensity readers or radioactivity counters respectively, may be used to quantify the labelled compound or the labelled metabolites thereof in the luminal and abluminal compartments.

The invention in addition relates to the use of the device according to the invention with a view to studying the physiopathology of a disease, testing molecules developed with a preventive, therapeutic or diagnostic purpose that target the cellular and molecular actors of the HEB, or testing the physical conditions or testing the protocols.

The term “studying the physiopathology of a disease” is understood to refer to studying the impact of diseases on the characteristics of the HEB, such as permeability, selectivity, electrical resistance, morphology of cells, etc. . . . . In this embodiment, the device then includes at least one model cell type of the pathology under study. In one embodiment, the expression of proteins and/or the functionality of transporters of the HEB such as for example the P-glycoprotein (P-gp) or the glucose transporter GLUT1 are compared in the devices comprising at least one model cell type of the pathology under study and the devices not including any model cell type of this pathology. The expression of proteins may be evaluated by Western Blot, ELISA, or gene expression (RTqPCR) techniques.

The term “testing molecules developed with a preventive, therapeutic or diagnostic purpose that target the cellular and molecular actors of the HEB” is understood to refer to studying the impact of these molecules on the HEB and possibly the passage thereof through the HEB. In this embodiment, at least one molecule to be tested is applied to the device according to the invention, and after a time period of exposure or incubation, the device is analysed in order to determine the changes that have been caused by the said at least one tested molecule. These changes may in particular relate to permeability, selectivity, electrical resistance, cell morphology etc. In particular, it is possible to study the outcome resulting from the addition of the molecule on the target by comparing the functionality and/or the expression of this target in devices in which the molecule to be tested has been applied to devices in which it has not been applied. It is also possible to study the passage of the molecule through the HEB in a device according to the invention and in particular by additionally adding at least one inhibitor of the HEB transporters or a physical condition.

The term “testing the physical conditions” is understood to refer to studying the impact of these conditions on the HEB and possibly their effect on the passage of compounds through the HEB. In this embodiment, at least one physical condition is applied to the device according to the invention. After a time period of exposure or incubation, the device is analysed to determine the changes that have been caused by the said at least one physical condition tested. These changes may in particular relate to permeability, selectivity, electrical resistance, cell morphology, etc. In particular, it is possible to study the outcome resulting from the addition of the physical condition by comparing it to a device to which the physical condition has not been applied. The term “physical condition” is understood in particular to refer to the use of waves such as magnetic, electromagnetic waves or even ultrasound waves.

The term “testing the protocols” is understood to refer to studying the impact of a treatment process on the HEB. In this embodiment, at least one treatment, that is to say a physical condition or a molecule, is applied to a compound as defined here above, and this compound is then brought into contact with the HEB. After a time period of exposure or incubation, the device is analysed in order to determine the changes that have been caused by the said treatment. These changes may in particular relate to permeability, selectivity, electrical resistance, cell morphology, etc. In particular, it is possible to study the outcome resulting from the treatment by comparing it to a device brought into contact with a compound that has not been treated.

The invention will now be described in more detail with the aid of examples taken into consideration on a non-exhaustive basis.

### FIGURES

FIG. 1: Schematic representation of the preparation of a device comprising cultures of primary cells according to the invention.

At day D1, the astrocytes and microglia are thawed and the endothelial cells and the pericytes are purified from mouse brains. The PBMCs are extracted from mouse peripheral blood.

At D3, the cell culture medium is renewed, ie replaced with new medium, with cessation of the effect of puromycin in the medium for endothelial cells.

At D5, the cell culture medium is renewed again.

At D8, the astrocytes and microglia, represented by dots “.”, are seeded in a culture dish. The culture medium for the other cells is renewed.

At D10, it is possible to observe the microglia cells.

At D11, the astrocytes and the microglia are seeded on the porous synthetic membrane.

At D12, the porous synthetic membrane is deposited in the culture dish in a manner such that the two astrocyte cultures are in contact, thus forming the abluminal compartment. Then the pericytes and the endothelial cells, represented by squares and circles (“□”, “o”) are seeded on the upper surface of the porous synthetic membrane, thus forming the luminal compartment. The treatment of the device with hydrocortisone is initiated.

At D15, the treatment of the device with hydrocortisone is completed, the PBMCs, represented by crosses “+”, are seeded into the luminal compartment. The model is ready for use.

FIG. 2: Paracellular permeability of FITC-Dextran on a device according to the invention comprising cultures of primary cells derived from mouse models of Alzheimer's Disease (AD) or wild type (WT) mice.

The test of permeability of the devices is performed with 4 kD FITC-dextran. The culture media are replaced by 1 mL of HBSS with Ca²⁺/Mg²⁺ in the abluminal compartment and 500 μL of FITC-dextran in the luminal compartment (that is to say 2.10⁻⁶ moles). Samples of 50 μL in the luminal and abluminal compartments are taken at 0 min, 10 min, 20 min, 30 min, 1 hr and 1 hr 30 min and deposited in the wells of a 96-well black plate, read on the Varioskan microplate reader (Thermo Scientific). The excitation wavelength (kex) of FITC-dextran is 485 nm and the emission wavelength (λem) is 515 nm.

FIG. 2 shows the results obtained for the abluminal compartment in the form of a curve. *p<0.05, **p<0.01 in relation to the control device which corresponds to a device without cells but coated with the same “coating” matrix as the other devices studied (AD versus WT).

The permeability of the device AD remains higher as compared to the device WT (46%), thus indicating a lower impermeability of the pathology related device as compared to the healthy device. The statistical test used is the Kruskal-Wallis test followed by the Dunn test for multiple comparisons.

FIG. 3: Paracellular permeability coefficient of FITC-dextran on a device according to the invention comprising cultures of primary cells from wild mice (WT).

The calculation of the permeability coefficient Pe is done by using this formula:

Pe=dQ/(dT*A*Co)

Pe: coefficient of permeability (cm/s)

dQ: quantity transported (mol)

dT: time of incubation (second)

A: surface area of the porous synthetic membrane (here 1.12 cm²)

Co: initial concentration (4.10⁻⁶ mol/cm³)

The determination of dQ is carried out based on the following calculation:

(Abluminal Fluorescence Intensity at 1 h)*2.10⁻⁶/(Luminal Fluorescence Intensity at T0)

The fluorescence intensity is proportional to the amount of FITC-dextran present in each compartment (abluminal and luminal).

The permeability coefficient is shown in FIG. 3 and was calculated after one hour. The results represent the mean±SEM (mean standard deviation) of the permeability coefficient of 3 to 4 devices in each group.*p<0.01 in relation to the control device which corresponds to a device without cells but covered with the same “coating” matrix as the device WT. The statistical test used is the Mann Whitney test.

FIG. 4: Functionality of the P-glycoprotein in the device according to the invention.

This functionality test is carried out with rhodamine 123, because it is known that this molecule as a substrate for the P-glycoprotein is expelled by the P-glycoprotein into the luminal compartment, which thus limits its passage through the device. The culture media are replenished: 1 mL in the abluminal compartment and 250 μL in the luminal compartment either containing or not containing the Zosuquidar inhibitor of P-glycoprotein P at 5 μM (Dantzig et al. 1996). The devices are incubated for 2 hrs in the incubator. Then, 250 μL of medium containing 2 μM rhodamine 123 with or without 5 μM Zosuquidar are added into the luminal compartment and incubated for 1 hr in the incubator. Samples of 50 μL in the luminal and abluminal compartments are taken just after the addition of the medium containing rhodamine 123 and after 1 hour of incubation at 37° C. The samples are placed in the wells of a 96-well black plate. The reading of the fluorescence intensity is done on the same apparatus as for the permeability test (described above). The λex of the rhodamine is 500 nm and the λem is 524 nm.

In FIG. 4, the results represent the mean±SEM of the fluorescence intensity after 1 hr of incubation of the cells with Rhodamine 123 (n=4 to 8 in each group). The passage of the rhodamine 123 through the device WT decreases by 72.6% as compared to the device without cells (**p<0.01). The presence of the specific P-glycoprotein inhibitor inhibits the efflux by 57.3%. The statistical test used is the Kruskal-Wallis test followed by the Dunn test for multiple comparisons.

FIG. 5: Trans-endothelial electrical resistance (TEER) in the murine devices according to the invention, having 12 and 24 wells.

In FIG. 5, the results represent the mean±SEM. For the 12-well format (FIG. 5A), n=16 controls and n=18 devices having the 12-well format. For the 24-well format (FIG. 5B), n=12 controls and n=74 devices having the 24-well format. The statistical test used is the Mann Whitney test: ****p<0.0001.

FIG. 6: Permeability coefficient of FITC-Dextran on murine devices according to the invention in 24- and 96-well format.

The coefficient of permeability is represented in FIG. 6 and in the same manner as in FIG. 3 after one hour. In FIG. 6, the results represent the mean±SEM. For the 24-well format (FIG. 6 A), n=12 controls and n=50 devices. For the 96-well format (FIG. 6B), n=8 controls and n=54 devices. The statistical test used is the Mann Whitney test: ****p<0.0001.

FIG. 7: Functionality of the P-glycoprotein in the murine device according to the invention in 24- and 96-well format.

In FIG. 7, the results represent the mean±SEM. For the 24-well format (FIG. 7A), n=12 controls and n=9 devices in the 24-well format without Zosuquidar and n=10 devices in the 24-well format with Zosuquidar. For the 96-well format (FIG. 7 B), n=7 controls and n=6 devices in the 96-well format without Zosuquidar and 9 devices in the 96-well format with Zosuquidar. The statistical test used is the Kruskal-Wallis test followed by a Dunn test for multiple comparisons: *p<0.05, **p<0.01, ***p<0.001.

FIG. 8: Coefficient of permeability of 8 molecules on murine devices according to the invention in 24 and 96-well format.

The molecules were added into the luminal compartment and incubated for a period of 48 hours. The luminal and abluminal media were then collected for the assay of the molecules. In FIG. 8 the different permeability coefficients calculated are represented (the results represent the mean±SEM), the results obtained for the 24-well format are shown in FIG. 8A, and the results obtained for the 96-well format in FIG. 8B.

FIG. 9: Trans-endothelial electrical resistance (TEER) and permeability coefficient of FITC-DEXTRAN in the commercially available device.

Represented in FIG. 9A is the Trans-endothelial Electrical Resistance (TEER) (n=14 controls and n=36 24-well devices). Represented in FIG. 9B is the permeability coefficient of FITC-DEXTRAN (n=8 controls and n=23 24-well devices). The results represent the mean±SEM. The statistical test used is the Mann Whitney test: ****p<0.0001.

FIG. 10: Functionality of P-glycoprotein in the commercially available device.

In FIG. 10, the results represent the mean±SEM of the fluorescence intensity after 1 hour of incubation of the cells with Rhodamine 123 (n=4 to 8 in each group). n=3 controls and n=4 commercially available 24-well devices with or without Zosuquidar. The statistical test used is the Kruskal-Wallis test followed by a Dunn test for multiple comparisons: *p<0.05.

FIG. 11: Trans-endothelial electrical resistance (TEER) in 24-well formats after cryopreservation.

Represented in FIG. 11 is the trans-endothelial electrical resistance (TEER) (n=3-4 controls and n=2-15 devices in the 24-well format). The 24-well format devices were cryopreserved for 7, 15 or 30 days, and then used 4, 5 or 6 days post-thawing. The results represent the mean±SEM. The statistical test used is the Mann Whitney test: *p<0.05, **p<0.01.

FIG. 12: Permeability coefficient of FITC-DEXTRAN in 24-well formats after cryopreservation.

In FIG. 8 the different permeability coefficients calculated are shown (n=8 controls and n=2-5 devices in the 24-well format). The results represent the mean±SEM). The devices in 24-well format were cryopreserved for 7, 15 or 30 days, and then used 4, 5 or 6 days post-thawing. The statistical test used is the Mann Whitney test: *p<0.05, **p<0.01.

FIG. 13: Trans-endothelial electrical resistance (TEER) in murine devices with immortalised cell lines according to the invention in 12- and 24-well format.

In FIG. 13, the results represent the mean±SEM. For the 12-well format (FIG. 13A), n=20 controls and n=20 devices in the 12-well format. For the 24-well format (FIG. 13B), n=10 controls and n=97 devices in the 24-well format. The statistical test used is the Mann Whitney test: ****p<0.0001.

FIG. 14: Permeability coefficient of FITC-dextran on murine devices with immortalised cell lines according to the invention in 12-, 24- and 96-well formats.

In FIG. 14, the results represent the mean±SEM. For the 12-well format (FIG. 14A), n=10 controls and n=6 devices in the 12-well format. For the 24-well format (FIG. 14B), n=17 controls and n=92 devices in the 24-well format. For the 96-well format (FIG. 14C), n=12 controls and n=63 devices in the 96-well format. The statistical test used is the Mann Whitney test: **p<0.01, ****p<0.0001.

FIG. 15: Functionality of P-glycoprotein on murine devices with immortalised cell lines according to the invention in 12-, 24- and 96-well format.

In FIG. 15, the results represent the mean±SEM. For the 12-well format (FIG. 15 A), n=4 controls and n=2 devices in the 12-well format with or without Zosuquidar. For the 24-well format (FIG. 15B), n=7 controls and n=9 and 12 devices in the 24-well format with or without Zosuquidar. For the 96-well format (FIG. 15C), n=12 controls and n=13 and 14 devices in the 96-well format with or without Zosuquidar.

The statistical test used is the Kruskal-Wallis test followed by a Dunn test for multiple comparisons: *p<0.05, **p<0.01, ***p<0.001.

### EXAMPLE 1: PREPARATION OF A DEVICE ACCORDING TO THE INVENTION

This device comprises primary cultures of endothelial cells, pericytes, and PBMCs harvested from mouse brains.

The development and preparation of this device involved going through the following technical steps:

The endothelial cells and pericytes were purified by using magnetic beads in order to exclude myelin and a Percoll gradient to dissociate the endothelial cells from the pericytes.

The PBMCs were extracted from the peripheral blood of the same mice on a Ficoll gradient identical to that used in human medical haematology and then recovered by centrifugation.

A primary co-culture stock of astrocytes and microglia was created as follows.

Primary mouse astrocyte and microglia cultures were obtained from brains of newborn mice between days D1 and D3. Cell dissociation of the brain tissue was performed mechanically. The astrocytes and microglia were selected by means of a selective culture medium. One week after the seeding of a newborn brain dissociated and cultured in a 75 cm2 Vial coated with Poly-L-Lysine, astrocytes forming a confluent mat were detached by using trypsin and cryopreserved. The thawing of a cone of cells was performed in a 25 cm² Vial, thus making it possible for the astrocytes to be used for the assembling of the device 72 hrs thereafter. These cells may be subcultured 3 times.

The cells were cultured in selective media for each cell type until a cell mat covering the entire surface of the selected culture dish was obtained.

The astrocytes and the microglia were seeded in a culture dish (30,000 cells per well for a 12-well dish).

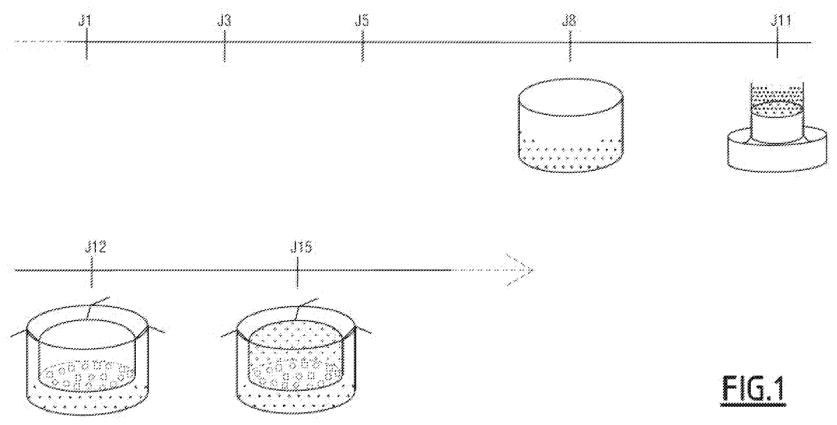

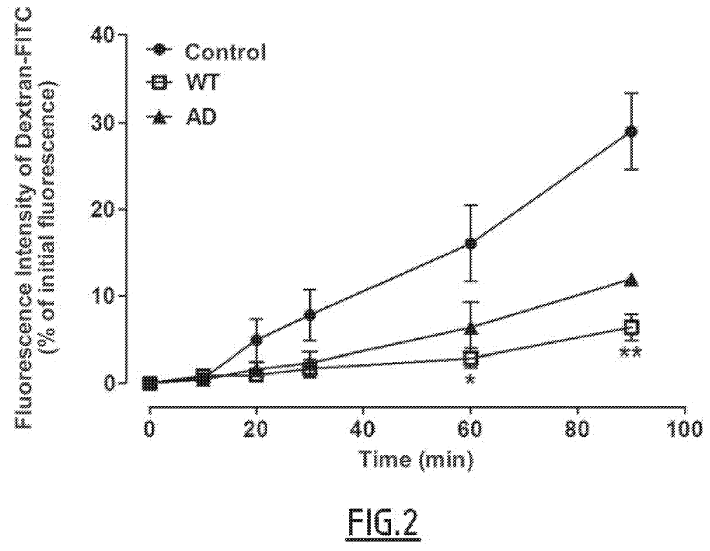

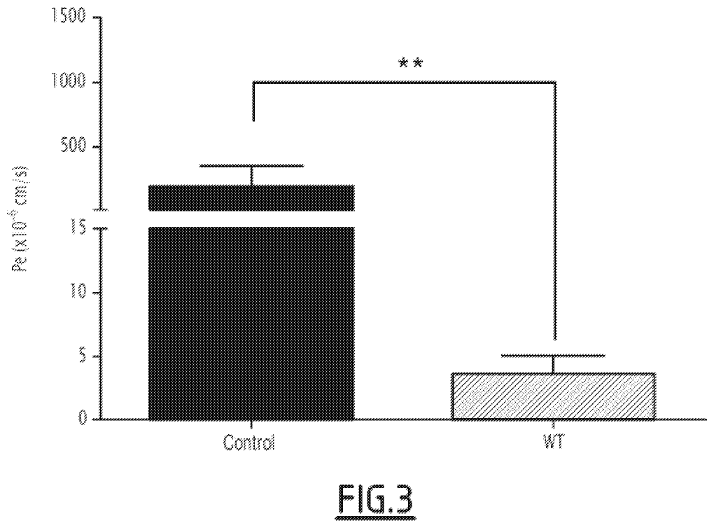

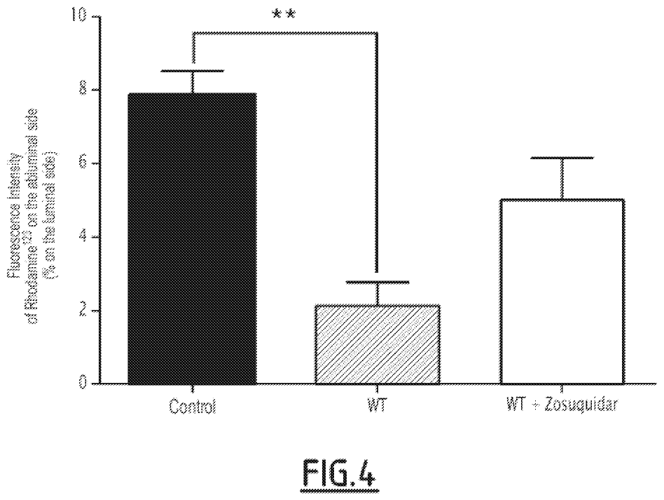

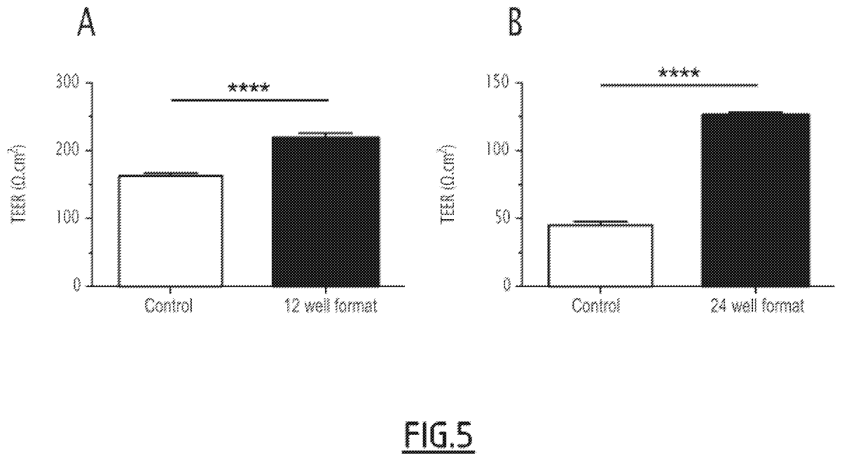

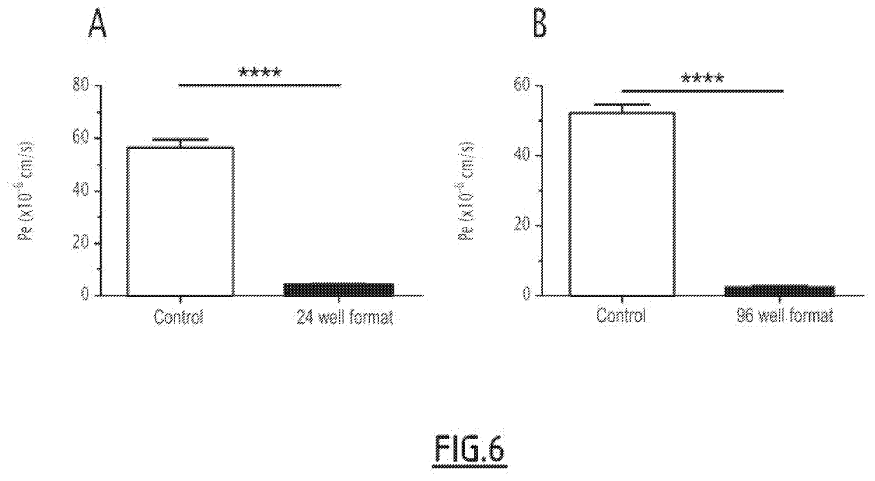

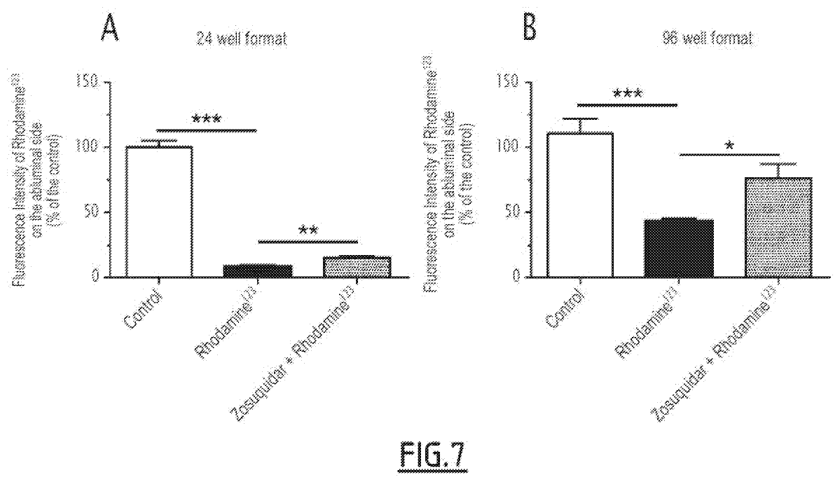

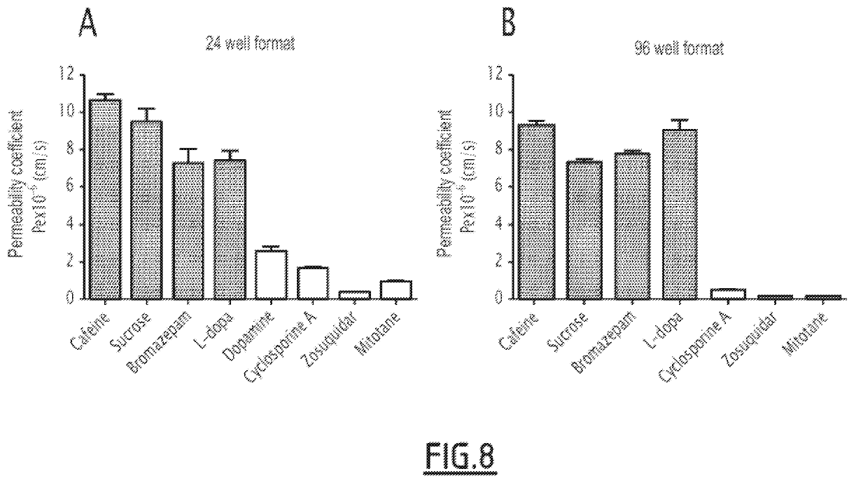

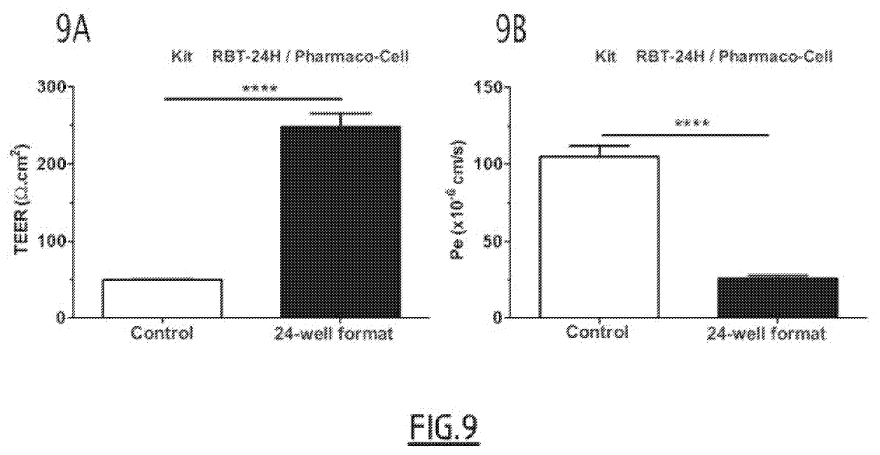

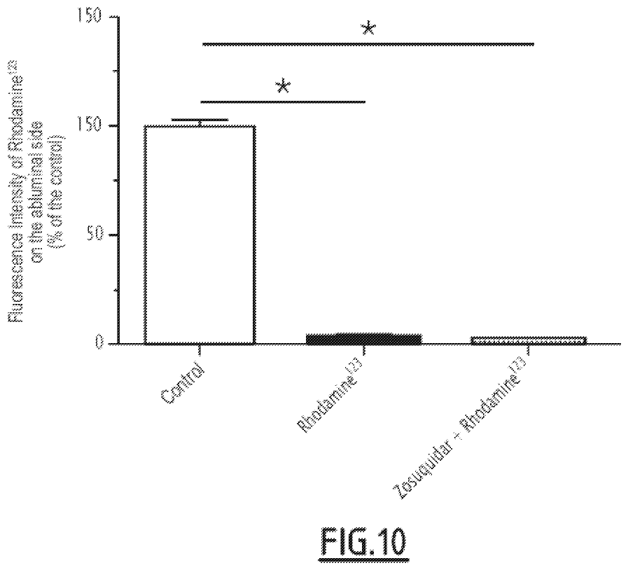

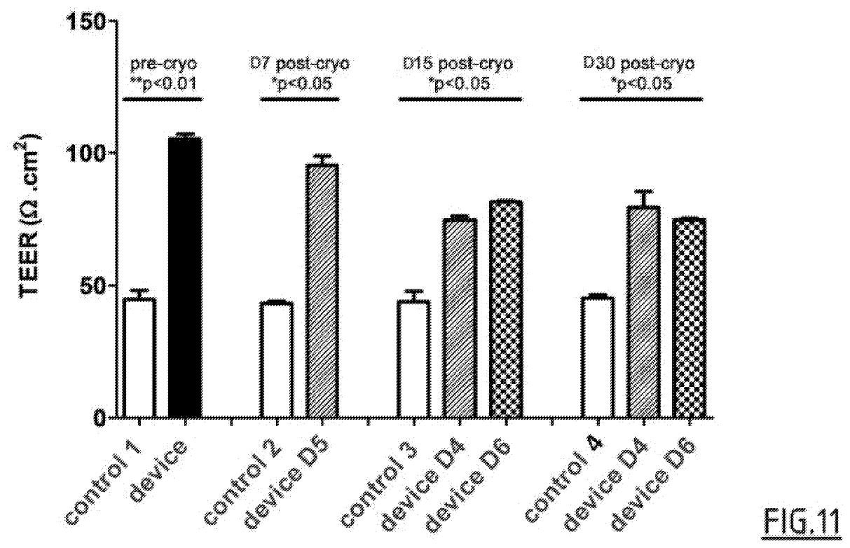

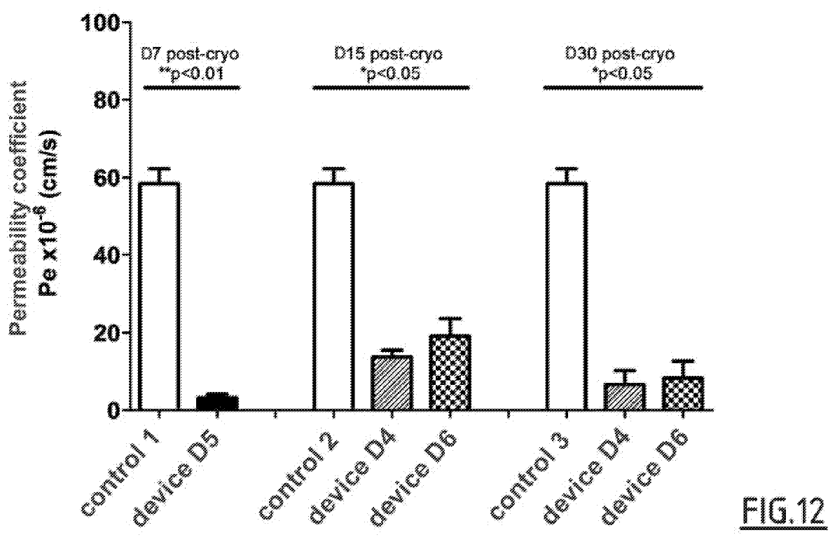

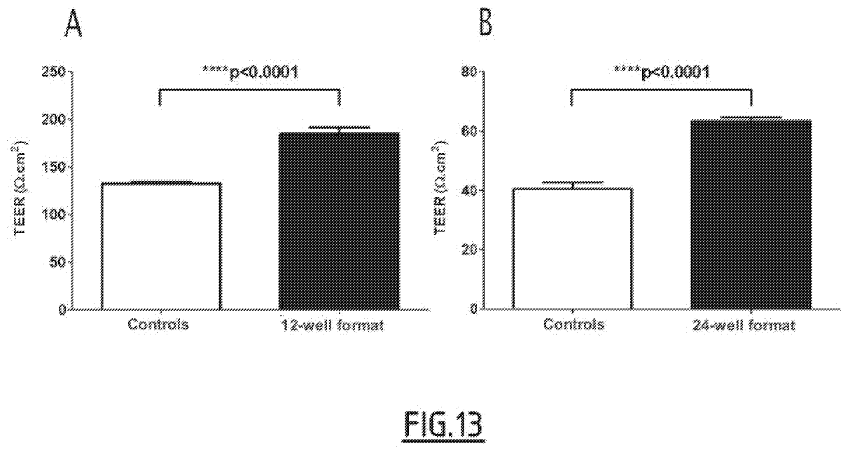

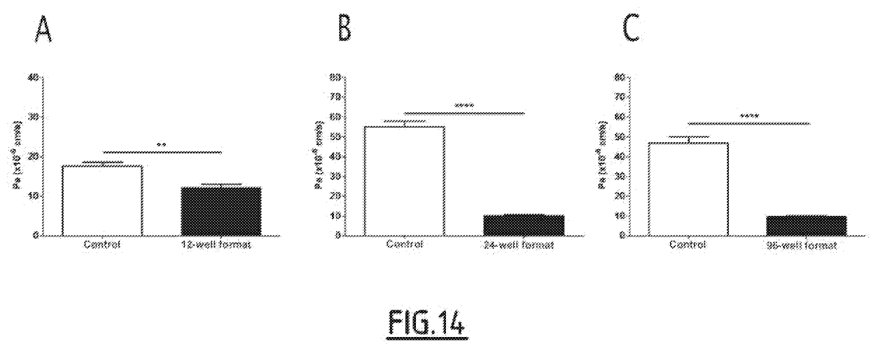

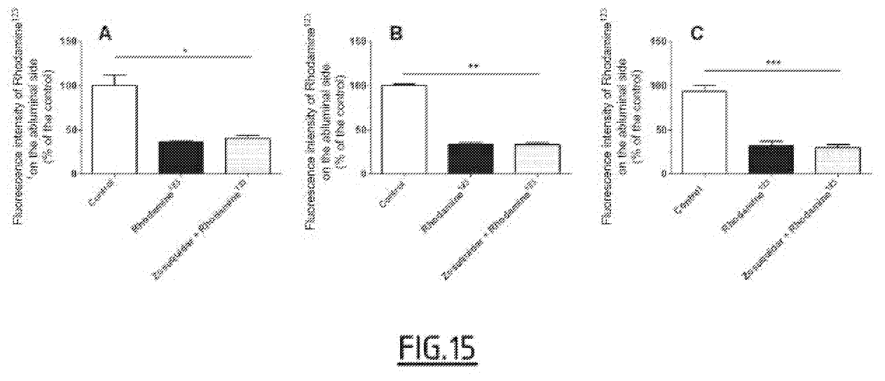

Then the astrocytes and microglia were also seeded under a porous synthetic membrane and incubated for 24 hours (see FIG. 1).

The membrane was deposited in the culture dish containing the astrocytes and the microglia in a manner such that the two astrocyte cultures were in contact, thereby forming the abluminal compartment. These cells thus model the glial cerebral parenchyma.

The endothelial cells and the pericytes were seeded onto the membrane (endothelial cells=10⁶ cells/membrane, and pericytes=350,000 cells/membrane in a 12-well dish).

The device was incubated for 72 hours in the presence of hydrocortisone so as to promote P-glycoprotein expression in the endothelial cells. In fact P-glycoprotein serves as an important efflux pump with respect to the functionality of the HEB.

After incubation, the model is ready. In particular, it is able to receive PBMCs.

### EXAMPLE 2: OBSERVATION BY MEANS OF IMMUNOLABELLING OF DEVICES MADE ACCORDING TO EXAMPLE 1

After the permeability and functionality tests, the porous synthetic membranes of the devices developed according to Example 1 are washed twice for a period of 5 min with 500 μL of Phosphate Buffered Saline (PBS). This is followed by addition of 500 μL of a paraformaldehyde solution (4% PFA) to the abluminal and luminal compartments for 15 min at ambient temperature. Two further 5-minute washes are carried out with 500 μL of PBS. The cells are then blocked and permeabilised with 500 μL of PBS/Triton 0.5%/Bovine Serum Albumin (BSA) 5% in the abluminal and luminal compartments for 1 hour at ambient temperature. On paraffin plastic film (parafilm) stretched over a petri dish, 30 μL of primary antibodies (Ad) are deposited. The membranes are then cut and subsequently deposited in a manner such that the cells are in contact with the primary antibodies (Ad). The antibodies used are anti-Zonula Occludens Protein 1 (ZO-1) antibodies (1/50 dilution, marker for tight junction proteins, used as marker for endothelial cells), anti-Alpha Smooth Muscle Actin (αSMA) (1/50 dilution, marker for pericytes), anti-von Willebrand Factor (vWF) (1/50 dilution, marker for endothelial cells) and anti-Glial Fibrillary Acidic Protein (GFAP) (1/100 dilution, marker for astrocytes). Incubation occurs over a period of one night at 4° C. in a humidity chamber. The next day, each membrane is gently placed in a 12-well dish with the side having the cells being studied on top and subjected to two 5 min washes with PBS with no agitation. 30 μL of Arthrobacter aurescens chondroitinase AC-II are deposited on a new paraffin plastic film before being incubated for 1 hour at ambient temperature with the membranes. The Ac II solution contains anti-mouse II secondary antibodies coupled to the fluorochrome Rhodamine Red-X (RRX) (red fluorescence) and anti-rabbit II secondary antibodies coupled to the fluorochrome Alexa 488 (green fluorescence) all diluted to 1:50. After 1 hr of incubation, the washes are done under the same conditions as above and then the membranes are incubated with 30 μL of DAPI solution (4,6-Diamino-2-Phenylindole) for 15 min at ambient temperature, protected from light, on paraffin plastic film in a wet chamber in order to mark the nuclei of the cells. The membranes are again recovered in order to undergo three 5 min washes with H2O UHQ to remove the salts. The membranes are then glued on a glass slide with DAPI glue in a manner such that the bonded side corresponds to that of the non-immunolabelled cells. A glass slide is then glued onto the membrane. The slides are observed under the epifluorescence microscope (Olympus BX 51).

The immunolabelling renders visible the cell types that make up the HEB model. In addition, it was observed that endothelial cells organise themselves into vessels with tight junctions being formed therebetween (ZO-1 tagging), thus spontaneously reproducing important characteristics of the HEB in vivo. The pericytes organise around the endothelial cells by establishing points of contact.

### EXAMPLE 3: USE OF A DEVICE ACCORDING TO THE INVENTION AS A MODEL OF THE HEB IN THE CASE OF CELLS DERIVED FROM MOUSE MODELS OF ALZHEIMER'S DISEASE (AD)

A device was prepared according to Example 1, here referred to as the Alzheimer device (device AD), in which the endothelial cells and pericytes were prepared from 4 to 8 week old Alzheimer mice (APPswePS1dE9, AD) and the astrocytes and microglia were prepared from wild mice (WT). This device was compared to a similar device formed completely from cells from wild mice (WT), here referred to as the wild device, and to a similar cell-free device, here referred to as the control device.

The results are shown in FIG. 2.

The presence of cells serves to decrease the passage of FITC-dextran (FIG. 2). In fact, it was observed that there was a significant 82% decrease in the passage of FITC-dextran through the wild device at 1 hour, and then a 78% decrease at 1.5 hours as compared to the control device without cells.

This difference in permeability is also observed with the device AD where there is a 60% decrease obtained at 1 hour and 1.5 hours as compared to the control. However, these results are not statistically significant.

In addition, although the observed difference in permeability between the AD and wild-type devices was not statistically significant, there was a 47% decrease observed in the passage of FITC-dextran through the wild-type device as compared to the device AD at 1.5 hours.

### EXAMPLE 4: TESTING THE PERMEABILITY AND FUNCTIONALITY OF A DEVICE ACCORDING TO THE INVENTION

In order to test the permeability of the device according to the invention, a device was prepared according to Example 1, and FITC-dextran was added to the luminal compartment of the device. The presence of FITC-dextran was detected and analysed by means of fluorescence.

In FIG. 3 it is noted that the coefficient of permeability is higher with the control device. A significant decrease in the permeability coefficient of 98% is observed for the device WT. This difference is statistically significant. It demonstrates the relative permeability of the device according to the invention.

In order to test the functionality of the device according to the invention, a device was prepared according to Example 1, and rhodamine 123 was added to the luminal compartment of the said device in the presence or absence of Zosuquidar. Indeed it is known that rhodamine 123 as a substrate for glycoprotein P is expelled by the glycoprotein P into the luminal compartment, which limits the passage thereof through the device. Zosuquidar is an inhibitor of P-glycoprotein, therefore its use should limit the efflux of rhodamine 123 into the luminal compartment.

In FIG. 4, it is observed that the passage of Rhodamine 123 to the abluminal compartment decreases by 72.6% for the wild device as compared to the control device (cell-free insert). These results are statistically significant. The presence of the specific inhibitor of glycoprotein P inhibits the efflux by 57.3%. These results demonstrate the functionality of the device according to the invention.

### EXAMPLE 5: VALIDATION OF A DEVICE ACCORDING TO THE INVENTION IN 24 AND 96 WELL PLATE FORMAT

The device preparation method based on these two new HEB formats is identical to that described in Example 1. Only the densities of each cell type had to be adapted.

TABLE 1
Cell densities of devices in 12, 24 and 96-well formats

| | Cell densities (cells/wells) | | | |
|---|---|---|---|---|
| | Astrocytes at | Astrocytes | Endothelial | |
| Format | bottom of well | below insert | cells | Pericytes |
| 12 wells | 30,000 | 74,000 | 1,000,000 | 350,000 |
| 24 wells | 15,000 | 21,765 | 350,000 | 117,000 |
| 96 wells | 2,500 | 9,450 | 250,000 | 85,000 |

The relevance of these formats was validated by using four tests: trans-endothelial electrical resistance (TEER), paracellular permeability, Glycoprotein G (P-gp) functionality and selectivity with respect to 8 molecules.

1—Trans-Endothelial Electrical Resistance (TEER)

The Trans-endothelial Electrical Resistance was only measured on the 12 and 24-well format HEBs because the electrode is not suitable for the 96-well format. It is expressed in ohm·cm² taking into account the surface area of the insert: 1.12 and 0.33 cm² for the 12 and 24-well inserts, respectively. It was measured with an ohm meter (Millicell Electrical Resistance System-2, Millipore—[Molsheim] France) using two STX01 electrodes: the larger one is placed in the abluminal compartment and the smaller one in the luminal compartment. The system carrying the two electrodes is connected to the ohm meter to measure the electrical resistance between the two compartments. The value displayed on the device is expressed in ohm and then multiplied by the surface area of the insert to obtain the results in ohm·cm².

FIG. 5 shows that the TEER is significantly increased in devices according to the invention in relation to the control (that is to say a device without any cells).

2—Paracellular Permeability

As in Example 4, the passage of FITC-dextran was studied and the permeability coefficient (Pe) of this molecule as a control for paracellular permeability was calculated (FIG. 6).

The results obtained for both the 24-well and 96-well formats show a permeability coefficient of less than 4.10⁻⁶ cm/s as for the 12-well format (see FIG. 3).

3—Functionality of the P-Glycoprotein (P-Gp)

As in Example 4, the passage of rhodamine 123 in the presence or absence of Zosuquidar was studied.

The P-gp pumps are functional in both 24- and 96-well format devices because Rhodamine123 is effluxed significantly towards the luminal side and therefore passes very little on to the abluminal side. This functionality is indeed inhibited by Zosuquidar known as a specific inhibitor of P-gp. This shows that as with the 12-well format, the endothelial cells expressing P-gp are indeed polarised, the addition of Rhodamine123 into the abluminal compartment results in passage thereof to the luminal side that is comparable to cell-free HEBs.

The results are shown in FIG. 7.

4—Selectivity with Respect to 8 Molecules

On the 24 and 96-well devices, the passage of 8 molecules known to either be able or unable to pass through the HEB was studied.

Dopamine (DA), Levodopa (L-DOPA), Bromazepam (BROMO), Caffeine (CAF), Sucrose (SUC), Cyclosporin A (CYCLA), Zosuquidar (ZOSU), and Mitotane (MITO) were tested at a physiological and/or therapeutic concentration known in the literature in humans.

Indicated in Table 2 here below are the concentrations chosen for each molecule, as well as whether they are known to be able to pass through the HEB (+) or unable to pass through the HEB (−).

TABLE 2
Concentration of molecules used in the test and ability to pass through the HEB

| | Name of Molecule | | | | | | | |
|---|---|---|---|---|---|---|---|---|
| | CAF | SUC | BROMO | L-DOPA | DA | CYCLA | ZOSU | MITO |
| Passage | + | + | + | + | − | − | − | − |
| through the | | | | | | | | |
| HEB | | | | | | | | |
| Concentration | 40 | 2000 | 1 | 160 | 50 | 70 | 0.3 | 20 |
| (μg/mL) in | | | | | | | | |
| the insert | | | | | | | | |

The molecules were added to the luminal compartment at the indicated concentration and incubated for a period of 48 hours. The luminal and abluminal media were then removed for the assay of the molecules.

For each molecule, the coefficient of permeability was calculated. The results are shown in FIG. 8. The results validate the passage of these molecules through the devices of the invention which serve here as a model of the murine HEB, that is to say the first 4 molecules pass through whereas the last 4 do not (hence a very low coefficient of permeability for the last 4 molecules).

### EXAMPLE 6: COMPARISON OF THE DEVICE ACCORDING TO THE INVENTION WITH A COMMERCIALLY AVAILABLE DEVICE

The device in Example 1 in 24-well format was compared with a commercially available model (BBB Kit™ (RBT-24H) from Pharmaco-Cell®) which corresponds to a primary HEB model prepared from rat brain cells. In this commercially available device the endothelial cells are seeded on the insert, the pericytes under the insert, and the rat astrocytes at the bottom of the well.

The measurement of the TEER in this model shows a good transendothelial electrical resistance averaging 247±17.76 Ω·cm² (see FIG. 9 A). However, the permeability coefficient of FITC-dextran is higher than that of the device according to the invention (see FIG. 9 B) (25.850±2.308×10⁻⁶ cm/s versus the device of the invention 3.867±0.333×10⁻⁶ cm/s). The impermeability would therefore be better on the device of the invention by a factor of 6.7 as compared to that of the commercially available model.

Here again, the functionality of the commercially available model was evaluated by studying the passage of Rhodamine 123 in the presence or absence of the specific inhibitor of the P-gp glycoprotein, Zosuquidar.

The results show that the pump is present on both the luminal and abluminal sides, thus the cells are not properly polarised. Thus there is as much Rhodamine123 effluxed on the luminal side as on the abluminal side when it is deposited either in the luminal or in the abluminal compartment (97% efflux regardless of the side where it is deposited).

Thus, no effect is observed when Zosuquidar inhibits the P-gp pump in the commercially available model as shown in FIG. 10 contrary to the device according to the invention (see FIG. 7). Thus, the device according to the invention makes it possible to obtain a model with greater similarity to the HEB than the commercially available model that was tested here.

### EXAMPLE 7: CRYOPRESERVATION OF THE DEVICE ACCORDING TO THE INVENTION IN 24-WELL FORMAT

Cryopreservation of the device would provide the means for on-demand preparation and delivery of the frozen device to the client.

The device in 24-well format was cryopreserved with a CRYOSTOR® solution marketed at Sigma® (REF: C2874-100ML). A measurement of the TEER was performed before the cryopreservation at day D15 of the assembly of the device. CRYOSTOR volumes of 100 and 200 were selected for the cryopreservation of cells in the luminal and abluminal compartments, respectively.

Thawing was performed at days D7, D15, and D30 post-cryopreservation according to the following protocol and the impermeability of the thawed devices was investigated 4, 5, and 6 days after thawing (TEER and permeability coefficient of FITC-dextran).
On the day of thawing, preheat the complete Endogro™ culture medium with serum to 37° C.,
During the entire thawing process, do not touch the membrane of the insert with the pasteur pipettes or tips. Do not move the inserts. Handle and treat on a device by device basis,
Once the culture medium has been heated, immediately add 150 and 300 μL of Endogro™ complete culture medium with serum, on the luminal and abluminal sides, respectively,
Incubate for 3 hours at 37° C. under 5% CO₂.
After 3 hours, gently aspirate the culture media (CM) from the luminal and abluminal sides and add 200 and 500 μL of CM from the luminal and abluminal sides, respectively,
Incubate at 37° C. under 5% CO₂,
The day after thawing, replace the culture medium,
At days D4, D5 and D6, tests were carried out to measure the TEER and the coefficient of paracellular permeability. The results are shown in FIGS. 11 and 12 respectively.

Compared to the non-cryopreserved 24-well format device, it is observed that the TEER resistance after 7 days of freezing is of the same order of magnitude as that measured just before the freezing. For 15 and 30 days of freezing, an average decrease of 26% is observed but remains insignificant.

As for the paracellular permeability, it is found that the permeability coefficients are also of the same order of magnitude after 7 days of freezing as those obtained on the non-cryopreserved HEBs (3.278±0.925 versus 3.867±0.333 10⁻⁶ cm/s, respectively). For 15 and 30 days of freezing, the coefficient of permeability is between 6 and 20.10⁻⁶ cm/s.

### EXAMPLE 8: MURINE HEB DEVICE WITH IMMORTALISED CELL LINES

Cell lines of murine endothelial cells and murine pericytes after immortalisation of the primary cells were obtained by using a method already published in the literature (Burek et al. 2012).

The immortal nature of these lines was verified by performing a karyotype showing a change in the number of chromosomes. Under normal circumstances and conditions, in mice, 2n=40 chromosomes. In the immortal lines, the reading of 16 metaphasic plates shows several anomalies in the number of chromosomes validating immortalisation with 2n=39 to 77 chromosomes depending on the plates read.

These lines have a replication time of 48 hours. These lines may be cryopreserved. The culture media used are the same as those used for the primary cultures. Their immortal nature leads to very high cell adhesion.

The assembly of the device with these cells follows the following kinetics:
D1 seeding of astrocytes/microglia I under the insert and at the bottom of the well
D2 seeding of endothelial cells and pericytes on the insert
D3-D4 incubation of the model for 48 hours in the incubator
At D4 the device is ready for any experimentation.

Indicated in Table 3 below are the cell densities used for each cell type and for each format of the device (12-, 24-, and 96 wells).

TABLE 3
Cell densities of devices in 12-, 24-, and 96-well formats

| | Cell densities (cells/wells) | | | |
|---|---|---|---|---|
| | Astrocytes at | Astrocytes | Endothelial cell | Pericytes cell |
| Format | well bottom | under insert | line | line |
| 12 wells | 95,000 | 149,333 | 170,000 | 57,000 |
| 24 wells | 47,500 | 44,000 | 50,000 | 17,000 |
| 96 wells | 8,000 | 3,575 | 22,000 | 7,500 |

On the 12 and 24 well plate formats, the TEER was measured (see FIG. 13) and on all formats the paracellular permeability (see FIG. 14) and the functionality of the P-gp efflux pump (see FIG. 15) were evaluated.

For this HEB model, the co-immunolabelling was performed to render visible the expression of molecular markers specific to each cell type. The inventors thus observed that the immortalised endothelial cells express the following in the device:
P-glycoprotein,
the tight junction proteins: ZO-1, Claudine-5,
the vonWillebrand factor (vWF),
the LRP-1 receptor, and
the transferrin receptor.

They do not express the pericyte markers α-SMA, NG2, platelet-derived growth factor β receptor (PDGFβR), indicating that the culture is pure. On the contrary, pericytes indeed express these 3 markers, and LRP-1, and do not express GFAP and vWF, indicating a pure culture of pericytes uncontaminated by endothelial cells and astrocytes.

Impermeability of the HEBs

FIG. 13 shows the results of the TEER for the 12- and 24-well plate formats.

The TEER on the 12-well format is quite comparable to that obtained on the device with primary cultures (see FIG. 5: 12-well TEER mean 218±7.17 Ω·cm² and here for the model with immortal cell lines the mean TEER is 184.60±6.65 Ω·cm²). For the 24-well format, the mean is 63.30±1.35 Ω·cm² while the HEB model with primary cultures had a mean TEER of 125.50±2.34 cm².

For the paracellular permeability, the permeability coefficient values for FITC-dextran are shown in FIG. 14 for each of the 3 formats (12-, 24-, and 96 wells, FIG. 14 A, B and C respectively). Regardless of the format used with the immortalised endothelial cell lines and pericytes in place of primary cultures, the results show that the devices are impermeable and the mean permeability coefficient is 12.15±0.92, 10.19±0.44, 9.66±0.50.10⁻⁶ cm/s for the 12-, 24-, and 96-well formats, respectively.

Functionality of the HEBs

The 12-, 24-, and 96 well plate formats are functional as they significantly limit the passage of Rhodamine¹²³ on the abluminal side by more than 60% (see FIG. 15). Contrary to the HEBs prepared with primary cultures, the specific Glycoprotein P inhibitor does not show any effect on all the formats, this may be related to a different efflux pump of the “Multi drug resistance” type very often encountered in cell lines.

### REFERENCE

Burek M, Salvador E, Förster C Y. Generation of an immortalised murine brain microvascular endothelial cell line as an in vitro blood brain barrier model. J Vis Exp. 2012 Aug. 29; (66):e4022. doi: 10.3791/4022
Dantzig A H, Shepard R L, Cao J, Law K L, Ehlhardt W J, Baughman T M, Bumol T F, Starling J J. Reversal of P-glycoprotein-mediated multidrug resistance by a potent cyclopropyldibenzosuberane modulator, LY335979. Cancer Res. 1996 Sep. 15; 56(18):4171-9.

1. A device comprising two compartments that are separated by a porous synthetic membrane, a luminal compartment comprising endothelial cells and pericytes, and an abluminal compartment comprising astrocytes.

2. A device according to claim 1, wherein the compartment comprising astrocytes additionally comprises microglia.

3. A device according to claim 1, wherein all of the cells originate from the same animal species.

4. A device according to claim 1, wherein one or more cell types are derived from primary cultures.

5. A device according to claim 1, wherein one or more cell types are derived from cultures of immortal cells.

6. A device according to claim 4, wherein the endothelial cells and pericytes are derived from primary cultures from an individual of the same age.

7. A device according to claim 4, wherein one or more of the said cell type(s) derived from primary cultures are disease model cell types.

8. A method for preparing a device according to claim 1, comprising the following steps:
a) seeding of astrocytes on one surface and/or on one side of the porous synthetic membrane;
b) inserting or depositing of the porous synthetic membrane on the said surface, in a manner such that if the porous synthetic membrane comprises astrocytes, then the side comprising the astrocytes is positioned to be facing the surface;
c) seeding of the luminal side of the porous synthetic membrane with endothelial cells and pericytes.

9. A method for preparing a device according to claim 1, comprising the following steps:
a) seeding of astrocytes on one side of the porous synthetic membrane;
b) seeding of the other side of the porous synthetic membrane with endothelial cells and pericytes, and optionally;
c) depositing of the porous synthetic membrane on one surface in a manner such that the side comprising astrocytes is positioned to be facing the surface.

10. Use of the device according to claim 1 as a model of the hemato-encephalic barrier (HEB).

11. The use according to claim 10 for testing the permeability of the model.

12. The use according to claim 10 for testing the permeability of the model to a compound comprising the following steps:
a) adding of the said compound to one of the compartments of the device;
b) incubation of the device;
c) detection and analysis of the presence of the said compound and/or its metabolites in the compartment where the addition of the said compound has not taken place.

13. The use according to claim 10 for studying the physiopathology of a disease, or testing molecules developed with a preventive, therapeutic or diagnostic purpose that target the cellular and molecular actors of the HEB, or testing physical conditions or testing protocols.

14. A device according to claim 6, wherein one or more of the said cell type(s) derived from primary cultures are disease model cell types.
